(19) United States
(12) Patent Application Publication
(10) Pub. No.: US 2019/0049955 A1
(43) Pub. Date: Feb. 14, 2019

(54) DRIVER STATE RECOGNITION APPARATUS, DRIVER STATE RECOGNITION SYSTEM, AND DRIVER STATE RECOGNITION METHOD

(71) Applicant: OMRON Corporation, Kyoto-shi (JP)

(72) Inventors: Tomohiro YABUUCHI, Kyoto-shi (JP); Tomoyoshi AIZAWA, Kyoto-shi (JP); Tadashi HYUGA, Hirakata-shi (JP); Hatsumi AOI, Kyotanabe-shi (JP); Kazuyoshi OKAJI, Omihachiman-shi (JP); Hiroshi SUGAHARA, Kyoto-shi (JP); Michie UNO, Kyoto-shi (JP); Koji TAKIZAWA, Kyoto-shi (JP)

(73) Assignee: OMRON Corporation, Kyoto-shi (JP)

(21) Appl. No.: 16/040,584

(22) Filed: Jul. 20, 2018

(30) Foreign Application Priority Data
Aug. 10, 2017 (JP) 2017-155260

(51) Int. Cl. G05D 1/00; B60Q 9/00; A61B 5/11; A61B 5/00; G06K 9/62; G06K 9/00; A61B 5/18

(52) U.S. Cl. CPC G05D 1/0061; B60Q 9/00; G05D 1/0088; A61B 5/1114; A61B 5/746; A61B 5/6893; G06K 9/6202; G06K 9/00832; G06K 9/00791; A61B 5/18; A61B 5/0077; A61B 5/1128

(57) ABSTRACT

A driver state recognition apparatus that recognizes a state of a driver of a vehicle having an autonomous driving system includes a state recognition data acquisition unit that acquires state recognition data of a state of a foot of a driver's seat of the vehicle, a leg state inference unit that infers a state of the driver's legs using the state recognition data acquired by the state recognition data acquisition unit, and a readiness determination unit that determines whether the driver is in a state of being able to immediately operate the pedal of the vehicle during autonomous driving, based on inference information from the leg state inference unit.

### CROSS-REFERENCES TO RELATED APPLICATIONS

This application claims priority to Japanese Patent Application No. 2017-155260 filed Aug. 10, 2017, the entire contents of which are incorporated herein by reference.

### FIELD

The disclosure relates to a driver state recognition apparatus, a driver state recognition system, and a driver state recognition method, and more particularly to a driver state recognition apparatus, a driver state recognition system, and a driver state recognition method that recognize a state of a driver of a vehicle that can drive autonomously.

### BACKGROUND

In recent years, development of autonomous driving technologies for vehicles has been actively pursued. It is required for a driver of a vehicle in which an autonomous driving system is installed to monitor the autonomous driving system even during autonomous driving, so that if, for example, an abnormality or failure occurs in the autonomous driving system, the autonomous driving system reaches the operating limit, or the autonomous driving control ends, the driver can smoothly take over a driving operation.

For example, in the following JP 4333797, a technology is disclosed for detecting drowsiness information such as the line of sight of the driver and the degree of opening of the eyelids from an image of the driver captured by a camera installed in the vehicle, calculating an actual degree of concentration using the drowsiness information, and reducing the traveling speed controlled by auto-cruise control in a case where the actual degree of concentration is lower than a required degree of concentration such as, for example, in a case where the driver's concentration decreases due to drowsiness.

As described above, if the driver dozes due to drowsiness during autonomous driving, the driver cannot monitor the autonomous driving system responsibly. As states in which the driver cannot monitor the autonomous driving system responsibly, various states other than the above dozing state are conceivable. In particular, during autonomous driving, the driver can adopt a posture that is not possible during manual driving, and thus the importance of recognizing the state of the driver during autonomous driving further increases. For example, it is necessary to appropriately recognize whether the driver is adopting a posture that enables the driver to immediately cope with manual driving even if the driving mode is switched from the autonomous driving mode to the manual driving mode because of the occurrence of a failure in the system during autonomous driving. However, in the monitoring method using the driver's drowsiness information disclosed in JP 4333797, there is a problem in that the posture of the driver cannot be appropriately recognized.

JP 4333797 is an example of background art.

### SUMMARY

One or more aspects have been made in view of the above circumstances, and one or more aspects may provide a driver state recognition apparatus, a driver state recognition system, and a driver state recognition method that can accurately recognize a driver's posture so as to enable the driver to promptly take over a driving operation, particularly a pedal operation, even during autonomous driving.

In order to achieve the above object, a driver state recognition apparatus according to one or more aspects is a driver state recognition apparatus for recognizing a state of a driver in a vehicle provided with an autonomous driving system, the driver state recognition apparatus including:
a state recognition data acquisition unit configured to acquire state recognition data of a vicinity of a foot of a driver's seat of the vehicle,
a leg state inference unit configured to infer a state of leg of the driver using the state recognition data acquired by the state recognition data acquisition unit; and
a readiness determination unit configured to determine, based on inference information from the leg state inference unit, whether the driver is in a state of being able to immediately operate a pedal of the vehicle during autonomous driving.

According to the above driver state recognition apparatus, the state of the legs of the driver is inferred using state recognition data of the vicinity of the foot of the driver's seat of the vehicle, and it is determined, based on the inference information of the state of the legs, whether the driver can immediately operate the pedal of the vehicle during autonomous driving. In this manner, it is possible to accurately recognize whether the driver is adopting a posture that enables the driver to promptly take over the pedal operation even during autonomous driving, and it is possible to appropriately provide support such that takeover of manual driving from autonomous driving, particularly takeover of the pedal operation, can be performed promptly and smoothly even if a failure or the like occurs in the autonomous driving system during autonomous driving. Note that the pedal of the vehicle includes at least one of an accelerator pedal and a brake pedal.

In the driver state recognition apparatus according to one or more aspects, the readiness determination unit may determine whether the driver is in the state of being able to immediately operate the pedal of the vehicle during autonomous driving, based on a positional relationship between the leg of the driver and a floor of the vehicle, a positional relationship between the leg of the driver and the pedal of the vehicle, or a positional relationship between right and left legs of the driver.

According to the above driver state recognition apparatus, because it is determined whether the driver is in a state of being able to immediately operate the pedal of the vehicle during autonomous driving, based on the positional relationship between the legs of the driver, the positional relationship of the legs of the driver and the pedal of the vehicle, or the positional relationship between the right and left legs of the driver, the determination can be accurately performed in consideration of the state of the legs of the driver.

The driver state recognition apparatus according to one or more aspects may include a notification processing unit configured to perform predetermined notification processing based on a determination result of the readiness determination unit.

According to the above driver state recognition apparatus, by performing the predetermined notification processing based on the determination result of the readiness determination unit, it is possible to perform support in which the determination result is reflected with respect to the autonomous driving system.

Also, in the driver state recognition apparatus according to one or more aspects, if it is determined by the readiness determination unit that the driver is not in the state of being able to immediately operate the pedal of the vehicle, the notification processing unit may perform notification processing for prompting the driver to adopt an appropriate leg posture.

According to the above driver state recognition apparatus, if it is determined that the driver is not in the state of being able to immediately operate the pedal of the vehicle, notification processing for prompting the driver to adopt an appropriate leg posture is performed. In this manner, it is possible to prompt the driver to adopt an appropriate posture such that the driver keeps a posture that enables the driver to immediately operate the pedal even during autonomous driving.

Also, in the driver state recognition apparatus according to one or more aspects, if it is determined by the readiness determination unit that the driver is in the state of being able to immediately operate the pedal of the vehicle, the notification processing unit may perform processing for notifying the driver that a leg posture is appropriate.

According to the above driver state recognition apparatus, if it is determined that the driver is in a state of being able to immediately operate the pedal of the vehicle, the processing for notifying the driver that the driver is adopting an appropriate leg posture is performed. In this manner, the driver can recognize that the leg posture during autonomous driving is appropriate.

Also, in the driver state recognition apparatus according to one or more aspects, if it is determined by the readiness determination unit that the driver is not in the state of being able to immediately operate the pedal of the vehicle, the notification processing unit may perform processing for notifying an autonomous driving control apparatus that controls the autonomous driving system to continue rather than cancel autonomous driving.

According to the above driver state recognition apparatus, even if it is determined that the driver is not in the state of being able to immediately operate the pedal of the vehicle, it is possible to continue the autonomous driving control.

Also, the driver state recognition apparatus according to one or more aspects may include an information acquisition unit configured to acquire information arising during autonomous driving from the autonomous driving system, and the notification processing unit may perform the predetermined notification processing, based on the determination result of the readiness determination unit and the information arising during autonomous driving that is acquired by the information acquisition unit.

According to the above driver state recognition apparatus, the predetermined notification processing is performed according to the determination result of the readiness determination unit and the information arising during autonomous driving that is acquired by the information acquisition unit. In this manner, it is not required to needlessly perform various notifications to the driver and the like according to the situation of the autonomous driving system, and thus power and processing required for notification can be reduced.

Also, in the driver state recognition apparatus according to one or more aspects, the information arising during autonomous driving may include information for determining whether surroundings of the vehicle are in a safe state, and the notification processing unit may perform, if it is determined by the readiness determination unit that the driver is not in the state of being able to immediately operate the pedal of the vehicle, notification processing after changing a notification level for prompting the driver to correct a leg posture, according to whether the surroundings of the vehicle are in a safe state.

According to the above driver state recognition apparatus, if it is determined that the driver is not in a state of being able to immediately operate the pedal of the vehicle, it is possible to perform notification processing after changing the notification level for prompting the driver to correct his or her leg posture, according to whether the surroundings of the vehicle are in a safe state. As the information for determining whether the surroundings of the vehicle are in a safe state, it may be preferable that the information includes, for example, monitoring information of the surroundings of the vehicle. The monitoring information of the surroundings of the vehicle may be, for example, information notifying that the vehicle is being rapidly approached by another vehicle, or may be information indicating that the vehicle will travel a road where the functional limit of the system is envisioned, such as a narrow road with sharp curbs.

In a case where the surroundings of the vehicle are not in a safe state, for example, it may be preferable to raise the notification level and to perform a high-level notification for more strongly alerting the driver, such as by combining display, audio, vibration, and so on, for example, so that the driver adopts a posture in which he or she can immediately take over the pedal operation. On the other hand, if the surroundings of the vehicle are in a safe state, it may be preferable to lower the notification level and to perform a low-level notification by audio only, for example.

Also, in the driver state recognition apparatus according to one or more aspects, the information arising during autonomous driving may include take over request information for taking over manual driving from autonomous driving, and the notification processing unit may perform, if it is determined by the readiness determination unit that the driver is not in a state of being able to immediately operate the pedal of the vehicle, and the takeover request information is acquired by the information acquisition unit, notification processing for prompting the driver to take over a driving operation.

According to the above driver state recognition apparatus, if it is determined that the driver is not in a state of being able to immediately operate the pedal of the vehicle, and the takeover request information is acquired, it is possible to perform the notification processing for prompting the driver to take over the driving operation. The takeover request information may be information indicating that the vehicle has entered a takeover zone for taking over manual driving from autonomous driving, or information notifying that an abnormality or a failure has occurred in a part of the autonomous driving system. If the takeover request information is acquired, actual pedal operation is needed, and thus, for example, it may be preferable that notification is performed such that the driver takes over the driving operation by swiftly placing the foot on the pedal.

In the driver state recognition apparatus according to one or more aspects, the state recognition data acquisition unit may acquire, as the state recognition data, image data of a vicinity of a leg of the driver captured by a camera provided in the vehicle, and
the driver state recognition apparatus may further include:
a template image storage unit configured to store a template image for inferring the state of the leg of the driver, and the leg state inference unit may infer the state of the leg of the driver through template matching performed by using the image data of a vicinity of the leg of the driver acquired by the state recognition data acquisition unit and the template image read out from the template image storage unit.

According to the above driver state recognition apparatus, the state of the legs of the driver is inferred through template matching performed by using the image data of a vicinity of legs of the driver acquired by the state recognition data acquisition unit and the template image read out from the template image storage unit. Accordingly, it is possible to accurately infer the state of the legs of the driver based on the template image. Note that the state of the legs of the driver may be any of states of a portion from the ankles down, a portion from the knees down, and a portion from the thighs down.

In the driver state recognition apparatus according to one or more aspects, the state recognition data acquisition unit may acquire, as the state recognition data, leg detection data of the driver that is detected by a sensor provided at the foot of the driver's seat of the vehicle, and the leg state inference unit may infer the state of the leg of the driver using the leg detection data of the driver acquired by the state recognition data acquisition unit.

According to the above driver state recognition apparatus, the state of the legs of the driver is inferred by using the leg detection data of the driver acquired by the state recognition data acquisition unit. Accordingly, the state of the legs of the driver can be accurately inferred based on the leg detection data detected by the sensor. The sensor may be an object detection sensor such as a photoelectronic sensor, or other sensors such as a non-contact sensor that can detect the legs of the driver. Note that the state of the legs of the driver may be any of the state of a portion from the ankles down, a portion from the knees down, and a portion from the thighs down.

A driver state recognition system according to one or more aspects may include any of the above driver state recognition apparatuses, and a state recognition unit configured to recognize a state a foot of a driver's seat of the vehicle and output the state recognition data to the state recognition data acquisition unit.

According to the above driver state recognition system, the system is constituted to include the driver state recognition apparatus and a state recognition unit that recognize the state of the vicinity of the foot of the driver's seat of the vehicle and outputs the state recognition data to the state recognition data acquisition unit. In this configuration, it is possible to construct a system that accurately recognizes whether the driver is adopting a posture that enables the driver to promptly take over the pedal operation even during the autonomous driving, and that is able to appropriately provide support such that takeover of manual driving from autonomous driving can be performed promptly and smoothly, even in cases such as where a failure occurs in the autonomous driving system during autonomous driving. Note that the state recognition unit may be a camera that captures image data of the vicinity of the legs of the driver, a sensor that detects the legs of the driver in the vicinity of the foot of driver's seat, or a combination thereof.

Also, a driver state recognition method according to one or more aspects is a driver state recognition method for recognizing a driver state of a vehicle provided with an autonomous driving system, the method including:
a step of acquiring, from a state recognition unit configured to recognize a state of a vicinity of a foot of a driver's seat of the vehicle, state recognition data of the vicinity of the foot of the driver's seat,
a step of storing the acquired state recognition data in a state recognition data storage unit;
a step of reading out the state recognition data from the state recognition data storage unit;
a step of inferring a state of leg of the driver using the read state recognition data; and
a step of determining whether the driver is in a state of being able to immediately operate a pedal of the vehicle during autonomous driving, based on inference information of the inferred state of the leg of the driver.

According to the above driver state recognition method, the state of the legs of the driver is inferred using state recognition data of the vicinity of the foot of the driver's seat of the vehicle, and it is determined, based on the inference information of the state of the legs, whether the driver is in a state of being able to immediately operate the pedal of the vehicle during autonomous driving. In this configuration, it is possible to accurately recognize whether the driver is adopting a posture that enables the driver to promptly take over the pedal operation even during autonomous driving, and it is possible to provide support such that takeover of manual driving from autonomous driving, particularly takeover of the pedal operation, can be performed promptly and smoothly, even in cases such as where a failure occurs in the autonomous driving system during autonomous driving.

### BRIEF DESCRIPTION OF THE DRAWINGS

FIG. 1 is a block diagram illustrating a configuration of a relevant section of an autonomous driving system that includes a driver state recognition apparatus according to an embodiment 1.

FIG. 2 is a side view illustrating a vicinity of a driver's seat of a vehicle in which a driver state recognition system according to an embodiment 1 is installed.

FIG. 3 is a block diagram illustrating a hardware configuration of a driver state recognition apparatus according to an embodiment 1.

FIG. 4 is a block diagram illustrating an example of a readiness determination table stored in a storage unit of a driver state recognition apparatus according to an embodiment 1.

FIG. 5 is a flowchart illustrating a processing operation performed by a control unit in a driver state recognition apparatus according to an embodiment 1.

FIG. 6 is a block diagram illustrating a configuration of a relevant section of an autonomous driving system that includes a driver state recognition apparatus according to an embodiment 2.

FIG. 7 is a side view illustrating a vicinity of a driver's seat of a vehicle in which a driver state recognition system according to an embodiment 2 is installed.

FIG. 8 is a block diagram illustrating a hardware configuration of a driver state recognition apparatus according to an embodiment 2.

FIG. 9 is a diagram illustrating an example of a readiness determination table stored in a storage unit of a driver state recognition apparatus according to an embodiment 2.

FIG. 10 is a flowchart illustrating a processing operation performed by a control unit in a driver state recognition apparatus according to an embodiment 2.

### DETAILED DESCRIPTION

Hereinafter, embodiments of a driver state recognition apparatus, a driver state recognition system, and a driver state recognition method will be described based on the drawings. Note that the following embodiments are specific examples of the present invention and various technical limitations are applied, but the scope of the present invention is not limited to these embodiments unless particularly stated in the following description.

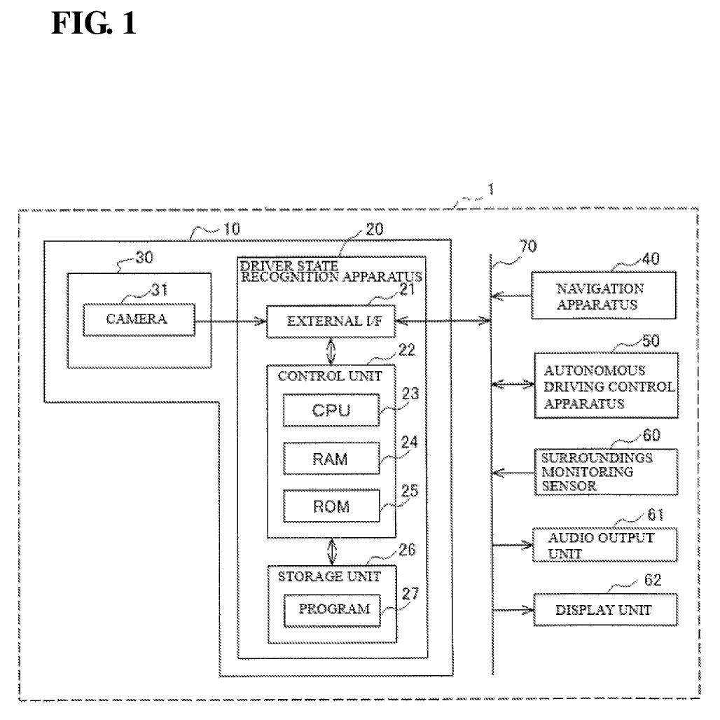

FIG. 1 is a block diagram showing a configuration of a relevant section of an autonomous driving system 1 that includes a driver state recognition apparatus 20 according to an embodiment 1.

The autonomous driving system is configured to include a driver state recognition apparatus 20, a state recognition unit 30, a navigation apparatus 40, an autonomous driving control apparatus 50, a surroundings monitoring sensor 60, an audio output unit 61, and a display unit 62, and these units are connected via a bus line 70.

The autonomous driving system 1 includes an autonomous driving mode in which the system, as the agent, autonomously performs at least a part of travel control including acceleration, steering, and braking of a vehicle and a manual driving mode in which a driver performs the driving operation, and the system is constituted such that these modes can be switched.

The autonomous driving mode in an embodiment is envisioned as being a mode in which the autonomous driving system 1 autonomously performs all of acceleration, steering, and braking and the driver copes with requests when received from the autonomous driving system 1 (automation level equivalent to so-called level 3 or greater), but the application of an embodiment is not limited to this automation level. Also, times at which the autonomous driving system 1 requests takeover of manual driving during autonomous driving include, for example, at a time of the occurrence of a system abnormality or failure, at a time of the functional limit of the system, and at a time of the end of an autonomous driving interval.

The driver state recognition apparatus 20 is an apparatus for recognizing a state of a driver of a vehicle including the autonomous driving system 1, and is an apparatus for providing support, by recognizing a state of a vicinity of a foot of the driver's seat and determining whether the legs of the driver is in a state of being able to immediately operate a pedal even during autonomous driving, so as to enable the driver to immediately take over manual driving, particularly to take over the pedal operation, if a takeover request of manual driving is generated from the autonomous driving system 1.

The driver state recognition apparatus 20 is configured to include an external interface (external I/F) 21, a control unit 22, and a storage unit 26. The control unit 22 is configured to include a Central Processing Unit (CPU) 23, a Random Access Memory (RAM) 24, and a Read Only Memory (ROM) 25.

The storage unit 26 is configured to include a storage apparatus that stores data with a semiconductor device, such as a flash memory, a hard disk drive, a solid-state drive, or other non-volatile memory or volatile memory. The storage unit 26 stores a program 27 to be executed by the driver state recognition apparatus 20 and the like. Note that part or all of the program 27 may be stored in the ROM 25 of the control unit 22 or the like.

The state recognition unit 30 for recognizing the state of the driver is configured to include a camera 31 that captures the vicinity of the foot of the driver's seat of the vehicle. The camera 31 is configured to include a lens unit, an image sensor unit, a light irradiation unit, an input/output unit and a control unit for controlling these units, which are not shown. The image sensor unit is configured to include an image sensor such as a CCD or CMOS sensor, filters, and microlenses. The light irradiation unit includes a light emitting device such as an LED, and may be an infrared LED or the like so as to be able to capture images of the state of the driver both day and night. The control unit is configured to include a CPU, a RAM, and a ROM, for example, and may be configured to include an image processing circuit. The control unit controls the image sensor unit and the light irradiation unit to irradiate light (e.g. near infrared light etc.) from the light irradiation unit, and performs control for capturing an image of reflected light of the irradiated light using the image sensor unit.

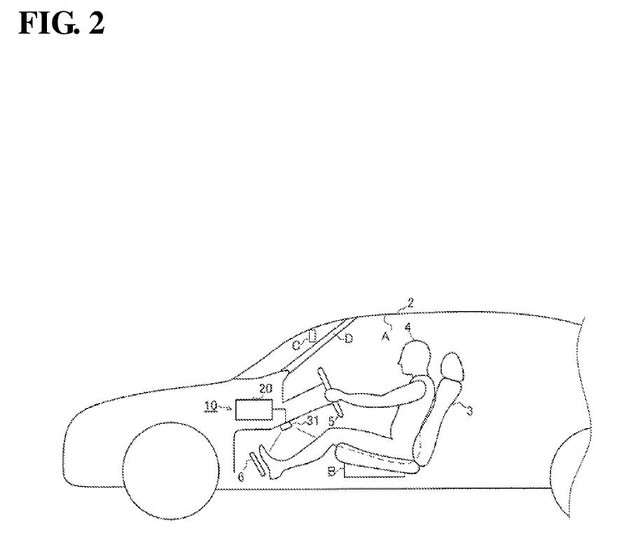

FIG. 2 is a side view showing a vicinity of a driver's seat of a vehicle 2 in which a driver state recognition system 10 according to an embodiment 1 is installed.

An attachment position of the camera 31 is not particularly limited, as long as it is a position at which a vicinity of a foot of a driver's seat 3 (a surrounding region of a pedal 6) in which a driver 4 is sitting can be captured. For example, the attachment position may be a cover portion underneath a steering wheel 5 as shown in FIG. 2. Also, as another attachment position, the camera 31 may be installed on a ceiling portion A, in a front portion of a sitting part of the driver's seat B, a position near a rear-view mirror C, or an A pillar portion D, as shown in FIG. 2, for example. The pedal 6 included at least one of a brake pedal and an accelerator pedal.

The number of cameras 31 may be one, or may also be two or more. The camera 31 may be configured separately (i.e. configured as a separate body) from the driver state recognition apparatus 20, or may be integrally configured (i.e. configured as an integrated body) with the driver state recognition apparatus 20.

The type of camera 31 is not particularly limited, and the camera 31 may be a monocular camera, a monocular 3D camera, or a stereo camera, for example. Image data captured by the camera 31 is sent to the driver state recognition apparatus 20. The driver state recognition system 10 is configured to include the driver state recognition apparatus 20, and the camera 31 serving as the state recognition unit 30.

The navigation apparatus 40 shown in FIG. 1 is an apparatus for provide the driver with information such as a current position of the vehicle and a traveling route from the current position to a destination, and is configured to include a control unit, a display unit, an audio output unit, an operation unit, a map data storage unit, and the like (not shown). Also, the navigation apparatus 40 is configured to be capable of acquiring a signal from a GPS receiver, a gyro sensor, a vehicle speed sensor, and the like (not shown).

The navigation apparatus 40 deduces information such as the road or traffic lane on which the vehicle is traveling and displays the current position on the display unit, based on the vehicle position information measured by the GPS receiver and the map information of the map data storage unit. In addition, the navigation apparatus 40 calculates a route from the current position of the vehicle to the destination and the like, displays the route information and the like on the display unit, and performs audio output of a route guide and the like from the audio output unit.

Also, some types of information such as vehicle position information, information of the road on which the vehicle is traveling, and scheduled traveling route information that are calculated by the navigation apparatus 40 are output to the autonomous driving control apparatus 50. The scheduled traveling route information may include information related to switching control between autonomous driving and manual driving, such as information on start and end points of autonomous driving zones and information on zones for taking over manual driving from autonomous driving.

The autonomous driving control apparatus 50 is an apparatus for executing various kinds of control related to autonomous driving of the vehicle, and is configured by an electronic control unit including a control unit, a storage unit, an input/output unit and the like (not shown). The autonomous driving control apparatus 50 is also connected to a steering control apparatus, a power source control apparatus, a braking control apparatus, a steering sensor, an accelerator pedal sensor, a brake pedal sensor, and the like (not shown). These control apparatuses and sensors may be included in the configuration of the autonomous driving system 1.

The autonomous driving control apparatus 50 outputs a control signal for performing the autonomous driving to each of the control apparatuses, performs autonomous traveling control of the vehicle (autonomous steering control, autonomous speed adjusting control, autonomous braking control and the like), and also performs switching control for switching between the autonomous driving mode and the manual driving mode, based on information acquired from each unit included in the autonomous driving system 1.

“Autonomous driving” refers to allowing the vehicle to autonomously travel along a road under control performed by the autonomous driving control apparatus 50 without the driver in the driver's seat performing the driving operation. For example, a driving state in which the vehicle is allowed to autonomously drive in accordance with a predetermined route to the destination and a traveling route that was automatically created based on circumstances outside of the vehicle and map information is included as autonomous driving. Then, if a predetermined cancelation condition of autonomous driving is satisfied, the autonomous driving control apparatus 50 may end (cancel) autonomous driving. For example, in the case where the autonomous driving control apparatus 50 determines that the vehicle in autonomous driving has arrived at a predetermined end point of an autonomous driving, the autonomous driving control apparatus 50 may perform control for ending autonomous driving. Also, if the driver performs autonomous driving cancel operation (for example, operation of an autonomous driving cancellation button, or operation of the steering wheel, acceleration, or braking or the like performed by the driver), the autonomous driving control apparatus 50 may perform control for ending autonomous driving. “Manual driving” refers to driving in which the driver drives the vehicle as the agent that performs the driving operation.

The surroundings monitoring sensor 60 is a sensor that detects target objects that exist in the vicinity of the vehicle. The target objects may include road markings (such as a white line), a safety fence, a highway median, and other structures that affect travelling of the vehicle and the like in addition to moving objects such as vehicles, bicycles, and people. The surroundings monitoring sensor 60 includes at least one of a forward-monitoring camera, a backward-monitoring camera, a radar, LIDER (that is, Light Detection and Ranging or Laser Imaging Detection and Ranging) and an ultrasonic sensor. Detection data of the target object detected by the surroundings monitoring sensor 60 is output to the autonomous driving control apparatus 50 and the like. As the forward-monitoring camera and the backward-monitoring camera, a stereo camera, a monocular camera or the like can be employed. The radar transmits radio waves such as millimeter waves to the surroundings of the vehicle, and detects, for example, positions directions, and distances of the target objects by receiving radio waves reflected by the target objects that exist in the surroundings of the vehicle. LIDER involves transmitting laser light to the surroundings of the vehicle and detecting, for example, positions, direction, and distances of the target objects by receiving light reflected by the target objects that exist in the surroundings of the vehicle.

The audio output unit 61 is an apparatus that outputs various kinds of notifications based on instructions provided from the driver state recognition apparatus 20 with sound and voice, and is configured to include a speaker and the like.

The display unit 62 is an apparatus that displays various kinds of notifications and guidance based on instructions provided from the driver state recognition apparatus 20 with characters and graphics or by lighting and flashing a lamp or the like, and is configured to include various kinds of displays and indication lamps.

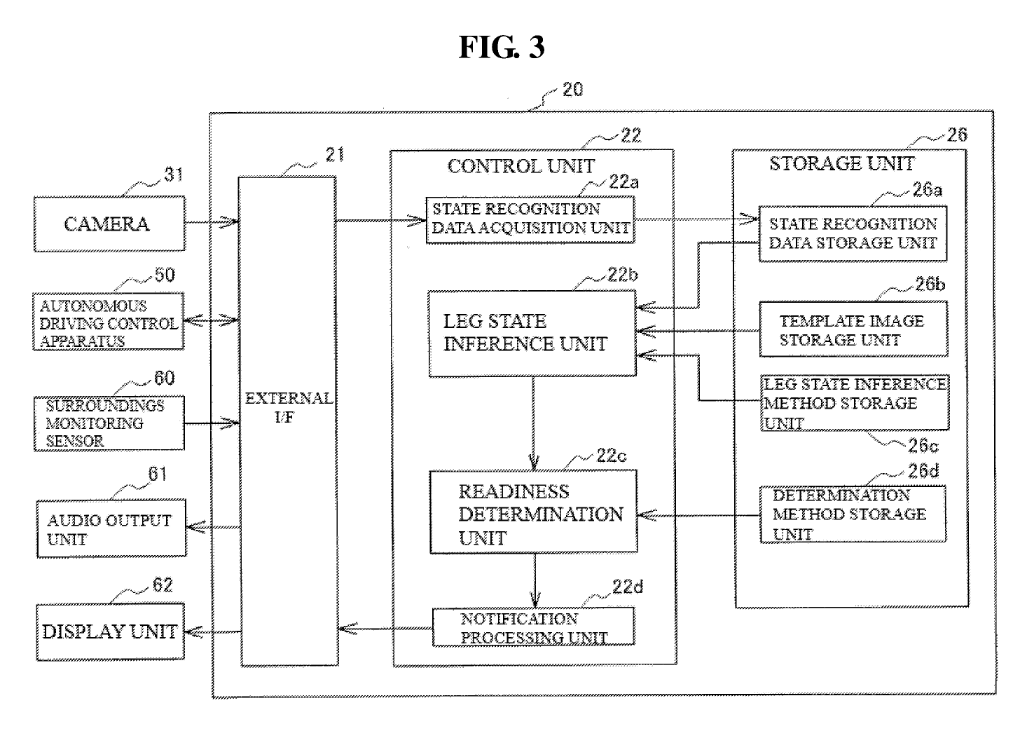

FIG. 3 is a block diagram showing a hardware configuration of the driver state recognition apparatus 20 according to an embodiment 1.

The driver state recognition apparatus 20 is configured to include the external interface (external I/F) 21, the control unit 22, and the storage unit 26. The external I/F 21 is connected to, in addition to the camera 31, each unit of the autonomous driving system 1 such as the autonomous driving control apparatus 50, the surroundings monitoring sensor 60, the audio output unit 61, the display unit 62, and is configured by an interface circuit and a connecting connector for transmitting and receiving a signal to and from each of these units.

The control unit 22 is configured to include a state recognition data acquisition unit 22a, a leg state inference unit 22b, and a readiness determination unit 22c, and may be configured to further include a notification processing unit 22d. The storage unit 26 is configured to include a state recognition data storage unit 26a, a template image storage unit 26b, a leg state inference method storage unit 26c, and a determination method storage unit 26d.

The state recognition data storage unit 26a stores image data of the camera 31 acquired by the state recognition data acquisition unit 22a.

The template image storage unit 26b stores template images that are used for template matching processing executed by the leg state inference unit 22b of the control unit 22.

The template images include images showing various states of the legs of the driver who is sitting in the driver's seat. Here, the states of the legs may include a positional relationship between the right and left legs and the floor, a positional relationship between the right and left legs and the pedal, and multiple different states in which these positional relationships are combined. Furthermore, the state of the legs may include a state that is known from a positional relationship between the left and the right legs. The template images include, for example, an image of a state in which both of the legs are on the floor near the pedal (within a predetermined range from the pedal), an image of a state in which one leg is in the vicinity of the pedal and the other leg is on the floor away from the pedal, and an image of a state in which the legs are crossed and one of the legs is on the floor near the pedal. Furthermore, the template images include images such as an image of a state in which both legs are on the floor at a position far away from the pedal (outside of the predetermined range from the pedal), an image of a state in which the legs are crossed and one of the legs is on the floor at a position far away from the pedal, an image of a state in which the driver sits cross-legged, an image of a state in which the driver sits with both knees up, and an image of a state in which no leg is captured (such as a state in which no driver is present). A state in which the both of the above legs are on the floor at a position far away from the pedal may include a state in which the knees are bent by 90 degrees or more.

The leg state inference method storage unit 26c stores, for example, a program that is executed by the leg state inference unit 22b of the control unit 22 for inferring the leg state using template matching method.

The determination method storage unit 26d stores, for example, a readiness determination table and a program that is executed by the readiness determination unit 22c of the control unit 22 and is for determining whether the driver is in a state of being able to immediately operate the pedal. For the readiness determination table, for example, data showing the relationship between the type of the template images and the readiness of the pedal operation can be used.

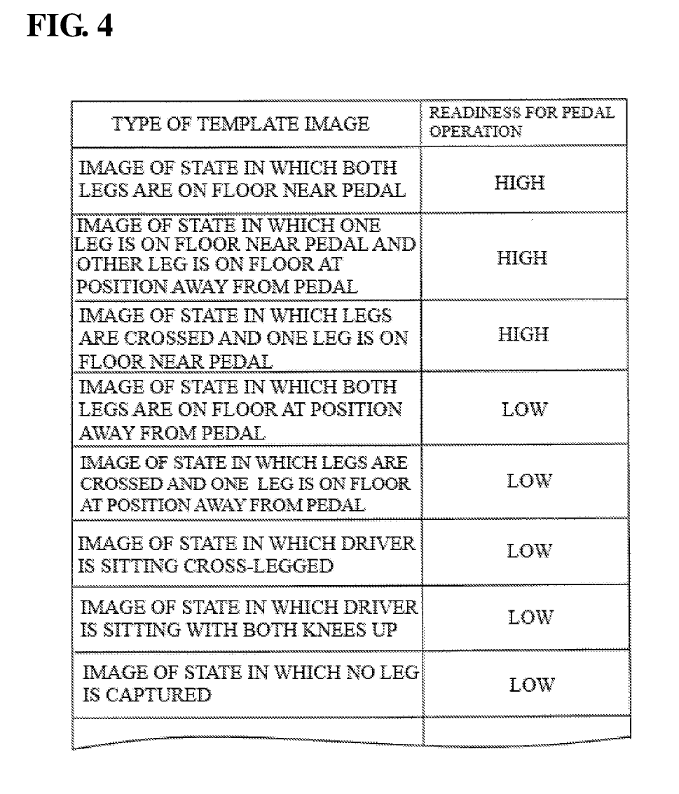

FIG. 4 shows an example of the readiness determination table stored in the determination method storage unit 26d. The readiness determination table stores the relationships between the types of the template images stored in the template image storage unit 26b and the readiness for pedal operation. The readiness of the pedal operation refers to the rapidity with which the driver can operate the accelerator pedal or the brake pedal if the autonomous driving system 1 stops operating for some reason and autonomous driving cannot be continued.

Accordingly, the state of high readiness for pedal operation means a state in which the driver can operate the pedal as soon as the driver needs to operate the pedal, and it is not necessary that the foot is placed on the pedal. The readiness of the pedal operation may be set in advance based on the positional relationship between each of the legs and the floor and the positional relationship between each of the legs and the pedal.

In the example shown in FIG. 4, a template image of the state in which both of the legs are on the floor near the pedal, a template image of the state in which one of the legs is on the floor near the pedal and the other leg is on the floor at a position away from the pedal, and a template image of the state in which the legs are crossed and one of the legs is on the floor near the pedal are associated with the readiness for pedal operation being high.

On the other hand, a template image of the state in which both legs are on the floor at a position far away from the pedal, a template image of the state in which the legs are crossed and one of the legs is on the floor at a position far away from the pedal, a template image of the state in which the driver sits cross-legged, a template image of the state in which the driver sits with both knees up, and a template image of the state in which no leg is captured are associated with the readiness for pedal operation being low.

Also, if at least one of the legs is on the floor near the pedal, it may be determined that the readiness for pedal operation is high. In addition, for example, it may be determined that the readiness is higher if, in the positional relationship of the right and left legs, the left leg is on the left side and the right leg is on the right side. It is possible to determine the readiness of the pedal operation corresponding to the leg state inferred by the leg state inference unit 22b (template image whose similarity is high) using this readiness determination table.

The control unit 22 is an apparatus that realizes functions of the state recognition data acquisition unit 22a, the leg state inference unit 22b, the readiness determination unit 22c, the notification processing unit 22d and the like, by performing, in cooperation with the storage unit 26, processing for storing various data in the storage unit 26, and by reading out various kinds of data and programs stored in the storage unit 26 and causing the CPU 3 to execute those programs.

The state recognition data acquisition unit 22a constituting the control unit 22 performs processing for acquiring an image of the vicinity of the foot of the driver's seat captured by the camera 31 and performs processing for storing the acquired image in the state recognition data storage unit 26a. The image that is acquired may be a still image, or may be a moving image. For example, the image of the vicinity of the foot of the driver's seat may be acquired at a predetermined interval after activation of the driver state recognition apparatus 20. Alternatively, the image of the vicinity of the foot of the driver's seat may be acquired after the driving mode has been switched to the autonomous driving mode.

The leg state inference unit 22b performs processing for inferring the state of the legs of the driver in the image, by reading out image data from the state recognition data storage unit 26a, and performing predetermined image processing on the image data based on template images that were read out from the template image storage unit 26b and a program that was read out from the leg state inference method storage unit 26c. More specifically, the leg state inference unit 22b performs template matching processing between the template images showing various states of the legs and the image captured by the camera 31, calculates the degree of similarity with these template images, extracts the template image whose degree of similarity is high, and infers the state of the legs of the driver.

The readiness determination unit 22c performs processing for determining whether the driver is in a state of being able to immediately operate the pedal, based on the program and the readiness determination table that were read out from the determination method storage unit 26d and the state of the legs of the driver that was inferred by the leg state inference unit 22b.

If the readiness determination unit 22c determines that the driver is not in a state of being able to immediately operate the pedal (the state in which the readiness is low), the notification processing unit 22d performs the notification processing for causing the audio output unit 61 and the display unit 62 to perform audio output and display output for prompting the driver to adopt a posture that enables the driver to immediately operate the pedal. The notification processing may be performed according to the inferred state of the legs. Also, the notification processing unit 22d may output a signal notifying to continue autonomous driving rather than cancel autonomous driving to the autonomous driving control apparatus 50.

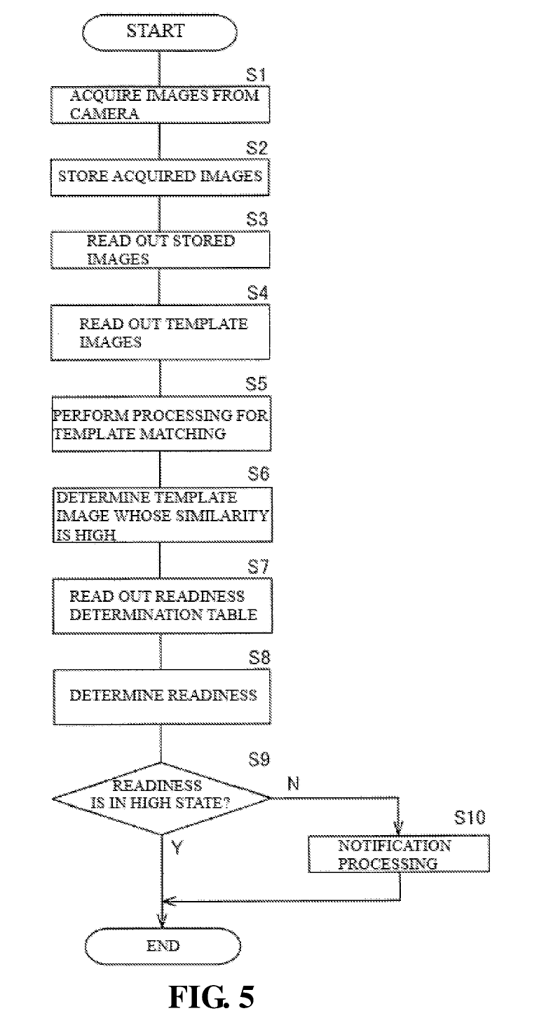

FIG. 5 is a flowchart a showing processing operation performed by the control unit 22 in the driver state recognition apparatus 20 according to an embodiment 1. Here, description will be given, assuming that the autonomous driving system 1 is set to the autonomous driving mode, that is, the vehicle is in a state of traveling under the autonomous driving control. The processing operations are repeatedly performed during the period in which the autonomous driving mode is set.

First, in step S1, processing for acquiring image data of the vicinity of the foot of the driver's seat captured by the camera 31 is performed. Image data may be acquired from the camera 31 one piece at a time, or multiple pieces of image data or pieces of image data over a certain time period may be collectively acquired. In the following step S2, processing for storing the acquired image data in the state recognition data storage unit 26a is performed, and thereafter the processing moves to step S3.

In step S3, the image data stored in the state recognition data storage unit 26a is read out, and then the processing moves to step S4. The image data may be acquired from the state recognition data storage unit 26a one piece at a time, or the multiple pieces of image data or pieces of image data over a certain time period may be collectively acquired. In step S4, the template images are read out from the template image storage unit 26b and the program and the like for inferring the state of the legs of the driver is read out from the leg state inference method storage unit 26c, and thereafter the processing moves to step S5.

In step S5, the template matching processing for calculating the degree of similarity and the like for the portion overlapping with the template images is performed on the read image data, while moving the template images showing various states of the legs one by one, and then the processing moves to step S6. For the template matching processing, various kinds of known image processing methods can be used.

In step S6, a template image whose degree of similarity is high is determined, and then the processing moves to step S7. In step S7, the readiness determination table, which is described in FIG. 4, is read out from the determination method storage unit 26d, and thereafter the processing moves to step S8. In step S8, the readiness that is associated with the type of the template image having a high degree of similarity is determined from the readiness determination table. For example, if the type of the template image that is determined to have a high degree of similarity in step S6 is the image of the state in which both of the legs are on the floor near the pedal, it is determined that the readiness is high. Also, if the type of the template image that was determined to have a high degree of similarity in step S6 is the image of the state in which the driver sits cross-legged, it is determined that the readiness is low.

In the following step S9, it is determined whether the readiness is in a high state, and if it is determined that the readiness is in the high state, that is, if it is determined that the driver is in a state of being able to immediately operate the pedal, the processing ends. Note that, in step S9, if the determination result showing that the readiness is high in step S8 has been detected for a certain time period, it may be determined that the readiness is in the high state. Also, in another embodiment, notification processing for notifying the driver that the driver is adopting an appropriate leg posture may be performed, such as, for example, by providing a correct posture notification lamp in the display unit 62 and turning on the appropriate posture notification lamp. Alternatively, a signal for notifying the driver that the driver is adopting an appropriate leg posture, that is, that the driver is adopting an appropriate posture for continuing autonomous driving, may by output to the autonomous driving control apparatus 50.

On the other hand, in step S9, if it is determined that the readiness is not in the high state, that is, if it is determined that readiness is in a low state, the processing moves to step S10. Note that, in step S9, if the determination result showing that the readiness is low in step S8 has been detected for a certain time period, it may be determined that the readiness is in the low state.

In step S10, the notification processing for prompting the driver to adopt an appropriate leg posture is performed. As notification processing, processing for outputting predetermined audio from the audio output unit 61 may be performed, or processing for displaying predetermined display on the display unit 62 may be performed. Also, notification according to the state of the legs may be performed. For example, if the state of the legs of the driver is cross-legged state, audio such as “please lower both legs to near the pedal” may be output. Also, in step S10, a signal notifying to continue autonomous driving rather than cancel autonomous driving may be output to the autonomous driving control apparatus 50.

According to the above driver state recognition system 10 according to an embodiment 1, a system is constituted by the driver state recognition apparatus 20 and the camera 31 that captures images of the vicinity of the foot of the driver's seat. Also, according to the driver state recognition apparatus 20, the state of the legs of the driver is inferred by the leg state inference unit 22b through template matching using the image data of the vicinity of the foot of the driver's seat captured by the camera 31 and the template images read out from the template image storage unit 26b. Then, it is determined, by the readiness determination unit 22c, whether the driver is in a state of being able to immediately operate the brake pedal or the accelerator pedal of the vehicle during autonomous driving based on the inference information of the state of the legs.

In this manner, it is possible to accurately recognize whether the driver is adopting a posture that enables the driver to immediately take over the pedal operation even during autonomous driving, and it is possible to appropriately provide support such that takeover of manual driving from autonomous driving, particularly takeover of the pedal operation, can be performed promptly and smoothly even if a failure or the like occurs in the autonomous driving system 1 during autonomous driving.

Also, according to the driver state recognition apparatus 20, if it is determined by the readiness determination unit 22c that the driver is not in a state of being able to immediately operate the pedal of the vehicle, that is, if it is determined that the readiness of the pedal operation is low, the notification processing for prompting the driver to adopt an appropriate leg posture is performed by the notification processing unit 22d. In this manner, it is possible to prompt the driver to correct his or her posture so as to keep a posture that enables the driver to immediately operate the pedal even during autonomous driving.

Note that, in the driver state recognition apparatus 20 according to an embodiment 1, template matching method is used as an image processing method for inferring the state of the legs of the driver performed by the leg state inference unit, but the image processing method for inferring the state of the legs of the driver is not limited to this method.

In another embodiment, a background difference method may be used that involves storing in advance a background image at the foot of the driver's seat in which the driver is not sitting in the driver's seat, and detecting the state of the legs of the driver by extracting the legs of the driver from the difference between that background image and the image captured by the camera 31.

Furthermore, a semantic segmentation method may be used that involves storing in advance a model of images including the leg portion of the driver who is sitting in the driver's seat with various leg postures obtained though machine learning by a learning machine, labeling the significance of each pixel in the image captured by the camera 31 using the learned model, and detecting the legs of the driver by extracting the leg portion of the driver.

Also, if a monocular 3D camera is used for the camera 31, the state of the legs of the driver may be detected using acquired information such as a detected position of each part or posture of the driver detected by the monocular 3D camera.

In addition, for detecting the state of the legs, it may be detected, with respect to each of the right and left foot, whether at least the foot is on the floor. Then, for detecting the state of the legs, it may be detected whether the leg is near the pedal by comparing the distance from the leg and the pedal with a predetermined threshold value.

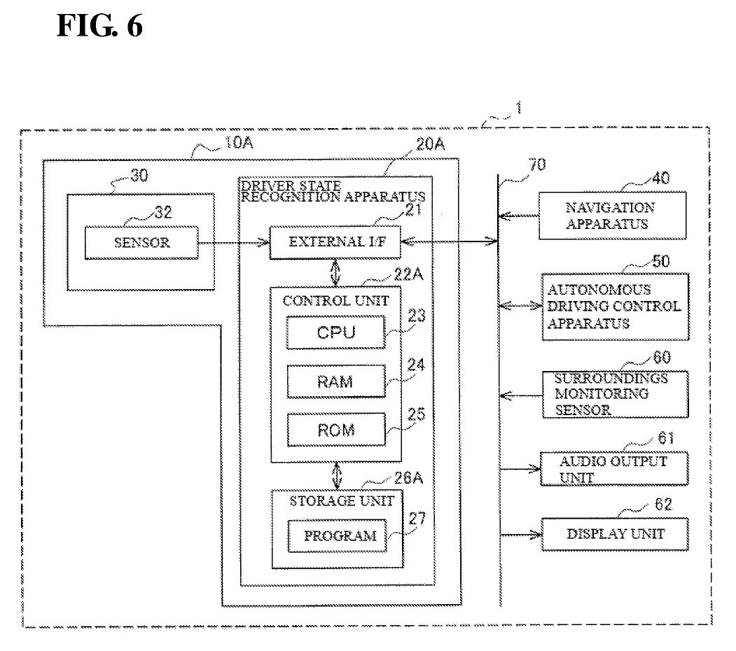

FIG. 6 is a block diagram showing a configuration of a relevant section of an autonomous driving system 1 that includes a driver state recognition apparatus 20A according to an embodiment 2. Note that constituent components that have the same functions as those of the autonomous driving system 1 shown in FIG. 1 are assigned the same numerals, and descriptions thereof are omitted here.

The camera 31 is used for the state recognition unit 30 in the driver state recognition system 10 according to an embodiment 1, but a driver state recognition system 10A according to an embodiment 2 differs greatly in that a sensor 32 is used for a state recognition unit 30A.

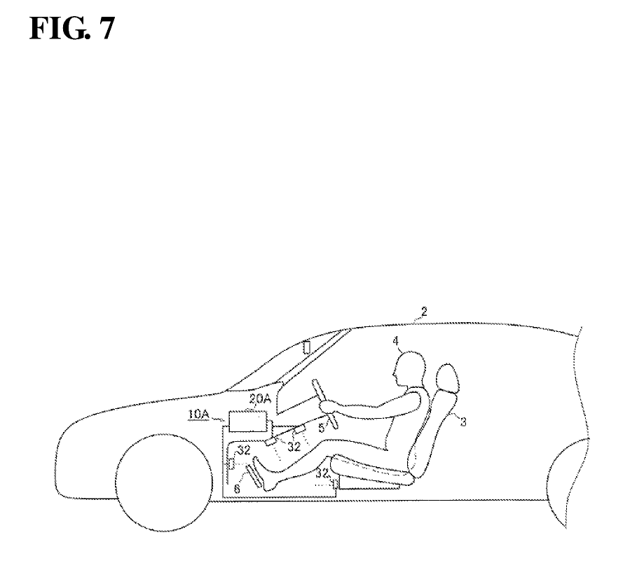

FIG. 7 is a side view showing a vicinity of a driver's seat of a vehicle 2 in which the driver state recognition system 10A according to an embodiment 2 is installed.

The sensor 32 is provided at the foot of the driver's seat 3 of the vehicle 2 and is an apparatus for detecting the legs of the driver 4. As the sensor 32, an object detection sensor such as a photoelectronic sensor can be employed. The photoelectronic sensor may be a transmission type photoelectronic sensor that has a light projector having a light projecting unit and a light receiver having a light receiving unit. Also, the photoelectronic sensor may be a retro-reflective photoelectronic sensor having a sensor body including a light projecting unit and a light receiving unit and a retro-reflective panel that reflects the light emitted from the light projecting unit, or may be a diffusive reflective photoelectronic sensor having a light projecting unit and a light receiving unit. Also, the photoelectronic sensor may be a distance measuring photoelectric sensor having a light projecting unit and a light receiving unit for position detection. For the light receiving unit for position detection, a position detection device or a two-piece diode may be used, and a distance to the detected object can be detected using the principle of triangulation with this configuration.

The number of sensors 32 is not particularly limited, but it is preferred to use multiple sensors 32 so as to detect the positions of each of the right and left legs of the driver 4.

Also, the attachment positions of the sensors 32 are not limited, but it is preferred to provide the sensors 32 in the vicinity of the foot of the driver's seat 3, for example, a back side portion of the pedal, a front portion of a sitting part of the driver's seat, a cover portion underneath a steering wheel and the like, so as to detect the positions of each of the right and left legs of the driver 4. Leg detection data detected by the sensors 32 is sent to the driver state recognition apparatus 20A. The driver state recognition system 10A is configured to include the driver state recognition apparatus 20A, and the sensors 32 serving as the state recognition units 30A.

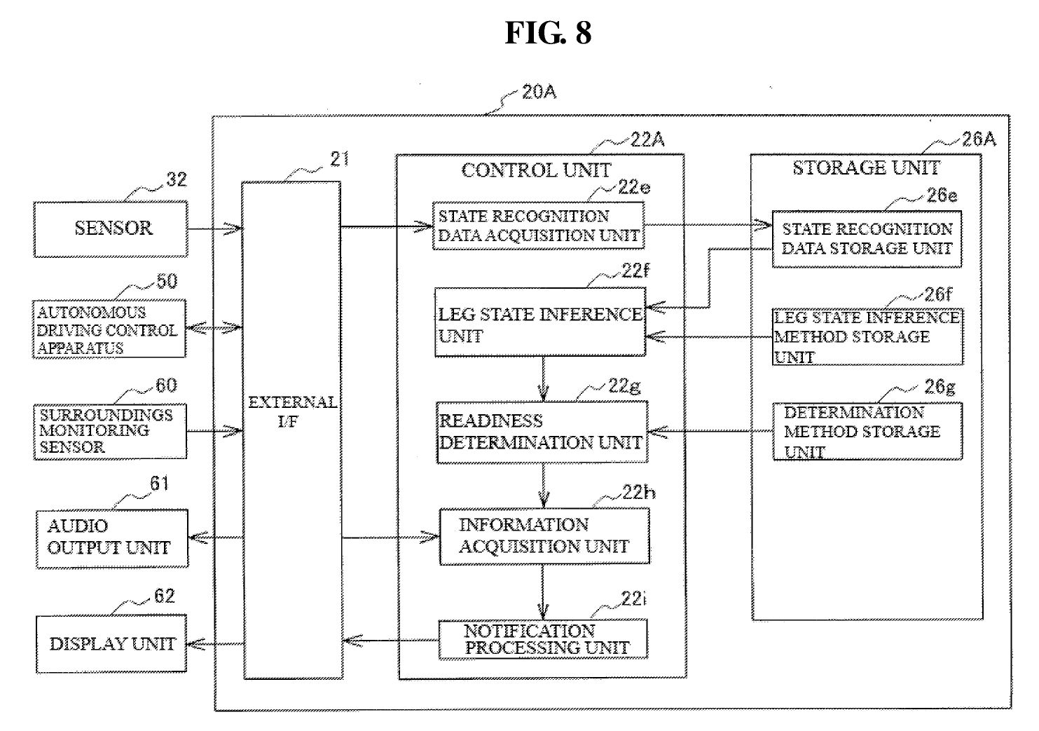

FIG. 8 is a block diagram showing a hardware configuration of the driver state recognition apparatus 20A according to an embodiment 2. Note that constituent components that have the same functions as those of the driver state recognition apparatus 20 shown in FIG. 3 are assigned the same numerals, and descriptions thereof are omitted here.

The driver state recognition apparatus 20A according to an embodiment 2 differs from the driver state recognition apparatus 20 according to an embodiment 1 in that the control unit 22A performs leg state inference processing, readiness determination processing, and notification processing based on data detected by the sensors 32.

The control unit 22A is configured to include a state recognition data acquisition unit 22e, a leg state inference unit 22f, a readiness determination unit 22g, an information acquisition unit 22h, and a notification processing unit 22i. A storage unit 26A is configured to include a state recognition data storage unit 26e, a leg state inference method storage unit 26f, and a determination method storage unit 26g.

The state recognition data storage unit 26e stores detection data of the legs of the sensors 32 acquired by the state recognition data acquisition unit 22e.

The leg state inference method storage unit 26f stores, for example, a program that is executed by the leg state inference unit 22f of the control unit 22A for inferring the state of the legs based on the detection data of the legs by the sensors 32.

The determination method storage unit 26g stores, for example, a program that is executed by the readiness determination unit 22g of the control unit 22A for determining whether the driver is in a state of being able to immediately operate the pedal and a readiness determination table. For the readiness determination table, for example, data showing a relationship between the detected position of the legs and the readiness of the pedal operation can be used.

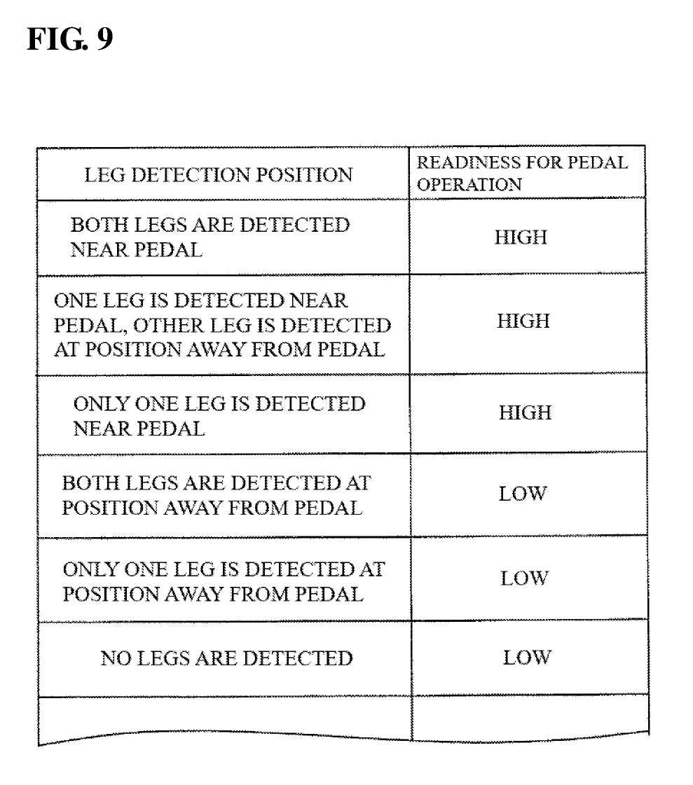

FIG. 9 shows an example of the readiness determination table stored in the determination method storage unit 26g. The detection position of the legs of the driver and the readiness of the pedal operation are stored in association with each other. In the example shown in FIG. 9, a case where both of the legs are detected near the pedal, a case where one of the legs is detected near the pedal and the other leg is detected at a position away from the pedal, and a case where only one of the legs is detected near the pedal are associated with the readiness for pedal operation being high.

Also, a case where both of the legs are detected at a position far from the pedal, a case where only one of the legs is detected at a position away from the pedal, and a case where no legs are detected are associated with the readiness for pedal operation being low. It is possible to determine the readiness of the pedal operation corresponding to the leg state inferred by the leg state inference unit 22f (detection position of the legs) using this readiness determination table.

The state recognition data acquisition unit 22e constituting the control unit 22 performs processing for acquiring the detection data of the legs of the driver detected by the sensors 32, and performs processing for storing the acquired detection data of the legs in the state recognition data storage unit 26e. The detection data of the legs provided from the sensors 32 can, for example, be acquired after the activation of the driver state recognition apparatus 20A, or can be acquired after the driving mode has been switched to the autonomous driving mode.

The leg state inference unit 22f reads out the detection data of the legs from the state recognition data storage unit 26e, and performs processing for inferring the state of the legs of the driver based on the program that is read out from the leg state inference method storage unit 26f. More specifically, the leg state inference unit 22f infers the leg positions of the driver based on the positions of the sensors 32 that detected the legs and the number of sensors 32 that detected the legs.

The readiness determination unit 22g performs processing for determining whether the driver is in a state of being able to immediately operate the pedal, based on the readiness determination table that was read out from the determination method storage unit 26g and the state of the legs of the driver that was inferred by the leg state inference unit 22f.

The information acquisition unit 22h acquires information arising during the autonomous driving from the units of the autonomous driving system 1. The information arising during the autonomous driving includes at least one of monitoring information of the surroundings of the vehicle that is detected by the surroundings monitoring sensor 60, and takeover request information for taking over manual driving from autonomous driving that is sent from the autonomous driving control apparatus 50.

If the readiness determination unit 22g determines that the driver is not in a state of being able to immediately operate the pedal (the state in which the readiness is low), the notification processing unit 22i performs, according to the information arising during autonomous driving that is acquired by the information acquisition unit 22h, processing for causing the audio output unit 61 and the display unit 62 to perform outputting processing for audio and display for prompting the driver to adopt a posture that enables the driver to immediately operate the pedal. Also, the notification processing unit 22i may output a signal for notifying the autonomous driving control apparatus 50 to continue autonomous driving rather than cancel autonomous driving.

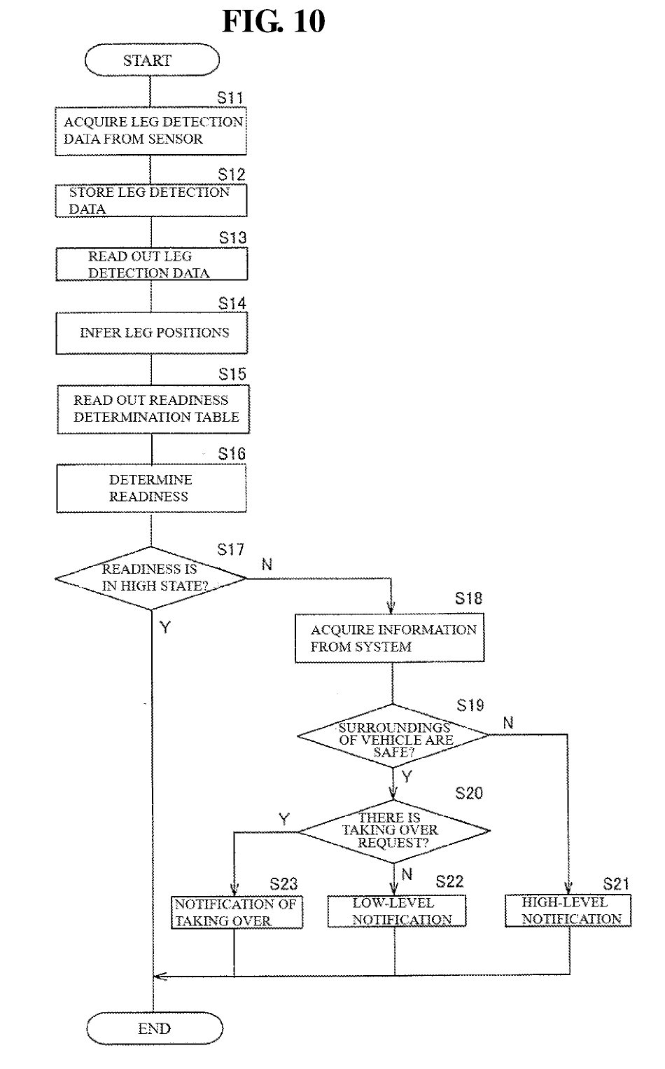

FIG. 10 is a flowchart showing a processing operation performed by the control unit 22A in the driver state recognition apparatus 20A according to an embodiment 2.

First, in step S11, the processing for acquiring the detection data of the legs of the driver detected by the sensors 32 is performed, in the following step S12, the processing for storing the acquired detection data of the legs in the state recognition data storage unit 26e is performed, in the following step S13, the detection data of the legs is read out from the state recognition data storage unit 26e, and then the processing moves to step S14.

In step S14, the processing for inferring the position of the legs of the driver from the read detection data of the legs is performed, and thereafter the processing moves to step S15. In the processing for inferring the position of the legs of the driver executed by step S14, the position of the legs is inferred using, for example, the positions of the sensors 32 that detected the detection data of the legs.

In step S15, the readiness determination table described in FIG. 9 is read out from the determination method storage unit 26g, and then the processing moves to step S16. In step S16, the readiness that is associated with the inferred positions of the legs of the driver is determined from the readiness determination table.

For example, if, with respect to the position of the legs inferred in step S14, both of the legs are detected near the pedal, it is determined that the readiness is high. On the other hand, if, with respect to the position of the legs inferred in step S14, only one of the legs is detected at a position away from the pedal, it is determined that the readiness is low.

In the following step S17, it is determined whether the readiness is in the high state, and if it is determined that the readiness is in the high state, that is, if it is determined that the driver is in a state of being able to immediately operate the pedal, and thereafter the processing ends. Note that, in step S17, if the determination result showing that the readiness is high in step S16 has been detected for a certain time period, it may be determined that the readiness is in the high state. Also, in another embodiment, notification processing for notifying the driver that the driver is adopting an appropriate leg posture may be performed, such as, for example, by providing an appropriate posture notification lamp in the display unit 62 and turning on the appropriate posture notification lamp. Alternatively, a signal for notifying the driver that the driver is adopting an appropriate posture, that is, that the driver is adopting an appropriate posture for continuing autonomous driving, may be output to the autonomous driving control apparatus 50.

On the other hand, in step S17, if it is determined that the readiness is not in the high state, that is, if it is determined that the readiness is in the low state, thereafter the processing moves to step S18. Note that, in step S17, if the determination result showing that the readiness is low in step S16 has been detected for a certain time period, it may be determined that the readiness is in the low state.

Information is acquired from the autonomous driving system 1 in step S18, and then the processing moves to step S19. This information includes the monitoring information of the surroundings of the vehicle that was detected by the surroundings monitoring sensor 60 and the takeover request information for taking over the manual driving that is output from the autonomous driving control apparatus 50. The takeover request information includes, for example, a system abnormality (failure) occurrence signal, a system functional limit signal, or an entry signal indicating entry to a takeover zone.

In step S19, it is determined whether the surroundings of the vehicle are in a safe state, based on the monitoring information of the surroundings of the vehicle that was acquired from the surroundings monitoring sensor 60. In step S19, if it is determined that the surroundings of the vehicle are not in a safe state, such as, for example, if it is determined that information indicating that another vehicle, a person, or another obstacle is detected in a certain range of the surroundings of the vehicle (any of forward, lateral, and backward), information notifying that another vehicle is rapidly approaching, or information indicating that the vehicle will travel on a road where the functional limit of the system is envisioned, such as a narrow road with sharp curbs was acquired, the processing moves to the high-level notification processing in step S21. In step S21, the high-level notification processing is performed with audio and display for causing the driver to swiftly adopt a posture that enables the driver to immediately operate the pedal, and then the processing ends. In the high-level notification processing, it is preferred that notification is performed in which display and audio are combined. Notification other than display or audio, such as, for example, applying vibrations to the driver's seat or the like may be added. Also, in step S21, a signal notifying to continue autonomous driving rather than cancel autonomous driving may be output to the autonomous driving control apparatus 50.

On the other hand, in step S19, if it is determined that the surroundings of the vehicle are in a safe state, thereafter the processing moves to step S20. In step S20, it is determined whether the takeover request information for taking over manual driving has been acquired from the autonomous driving control apparatus 50, that is, it is determined whether there is a takeover request. In step S20, if it is determined that there is no takeover request, the processing moves to low-level notification processing in step S22. In step S22, the low-level notification processing is performed for causing the driver to adopt a posture that enables the driver to operate the pedal, and then the processing ends. In the low-level notification processing, it is preferred to gently notify the driver, such as notification through display only, in order to achieve harmony between the autonomous driving and the driver.

On the other hand, in step S20, if it is determined that there was the takeover request, the processing moves to step S23. In step S23, takeover notification is performed by audio or display such that the driver immediately holds the steering wheel and takes over the driving, and then the processing ends.

In the above driver state recognition system 10A according to an embodiment 2, a system is constituted by the driver state recognition apparatus 20A and the sensors 32 that are provided at the foot of the driver's seat of the vehicle. Also, according to the driver state recognition apparatus 20A, the position of the legs of the driver is inferred by the leg state inference unit 22f based on the detection data of the legs of the driver detected by the sensors 32. Then, it is determined, by the readiness determination unit 22g, whether the driver is in a state of being able to immediately operate the brake pedal or the accelerator pedal of the vehicle during autonomous driving based on the inference information of the position of the legs.

In this manner, it is possible to accurately recognize whether the driver is adopting a posture that enables the driver to immediately take over the pedal operation even during autonomous driving, and it is possible to appropriately provide support such that takeover of manual driving from autonomous driving, particularly takeover of the pedal operation, can be promptly and smoothly performed even if a failure or the like occurs in the autonomous driving system 1 during autonomous driving.

Also, according to the driver state recognition apparatus 20A, the notification processing unit 22i performs notification processing for prompting the driver to adopt an appropriate posture according to the information arising during autonomous driving that is acquired by the information acquisition unit 22h. If surroundings monitoring information indicating that the vehicle-surroundings are not in the safe state is acquired, it is possible to raise the level of the notification performed by the notification processing unit 22i to more strongly alert the driver, such that the driver adopts a posture that enables the driver to immediately take over the pedal operation. Also, if the surroundings of the vehicle are safe, and there is no takeover request, it is possible to gently prompt the driver to correct his or her posture through the low-level notification. On the other hand, if there is the takeover request information, it is possible to notify the driver so as to adopt a posture for immediately taking over manual driving.

In this manner, it is not required to needlessly perform various notifications to the driver according to the state of the autonomous driving system 1, and thus power and processing required for the notification can be reduced. Also, at the time of failure occurrence or an operating limit of the system, or at the time of a request for taking over the manual driving, the time period until the driver operates the pedal and the takeover of the driving operation is completed can be shortened, thus enabling an appropriate notification that is gentle on the driver to be performed, according to the state of the autonomous driving system 1.

Note that, the information acquisition unit 22h may be provided in the driver state recognition apparatus 20 according to an embodiment 1, and instead of step S10 shown in FIG. 5, similar processing of steps S18 to s23 shown in FIG. 10, that is, notification processing, may be performed according to information arising during autonomous driving that is acquired by the information acquisition unit 22h.

1. A driver state recognition apparatus for recognizing a state of a driver of a vehicle provided with an autonomous driving system, comprising:
a state recognition data acquisition unit configured to acquire state recognition data of a vicinity of a foot of a driver's seat of the vehicle;
a leg state inference unit configured to infer a state of a leg of the driver using the state recognition data acquired by the state recognition data acquisition unit; and
a readiness determination unit configured to determine, based on inference information from the leg state inference unit, whether the driver is in a state of being able to immediately operate a pedal of the vehicle during autonomous driving.

2. The driver state recognition apparatus according to claim 1,
wherein the readiness determination unit determines whether the driver is in the state of being able to immediately operate the pedal of the vehicle during autonomous driving, based on a positional relationship between the leg of the driver and a floor of the vehicle, a positional relationship between the leg of the driver and the pedal of the vehicle, or a positional relationship between right and left legs of the driver.

3. The driver state recognition apparatus according to claim 1, further comprising:
a notification processing unit configured to perform predetermined notification processing based on a determination result of the readiness determination unit.

4. The driver state recognition apparatus according to claim 3,
wherein, if it is determined by the readiness determination unit that the driver is not in the state of being able to immediately operate the pedal of the vehicle, the notification processing unit performs notification processing for prompting the driver to adopt an appropriate leg posture.

5. The driver state recognition apparatus according to claim 3,
wherein, if it is determined by the readiness determination unit that the driver is in the state of being able to immediately operate the pedal of the vehicle, the notification processing unit performs processing for notifying the driver that a leg posture is appropriate.

6. The driver state recognition apparatus according to claim 3,
wherein, if it is determined by the readiness determination unit that the driver is not in the state of being able to immediately operate the pedal of the vehicle, the notification processing unit performs processing for notifying an autonomous driving control apparatus that controls the autonomous driving system to continue rather than cancel autonomous driving.

7. The driver state recognition apparatus according to claim 3, further comprising:
an information acquisition unit configured to acquire information arising during autonomous driving from the autonomous driving system,
wherein the notification processing unit performs the predetermined notification processing, based on the determination result of the readiness determination unit and the information arising during autonomous driving that is acquired by the information acquisition unit.

8. The driver state recognition apparatus according to claim 7,
wherein the information arising during autonomous driving includes information for determining whether surroundings of the vehicle are in a safe state, and
the notification processing unit performs, if it is determined by the readiness determination unit that the driver is not in the state of being able to immediately operate the pedal of the vehicle, notification processing after changing a notification level for prompting the driver to correct a leg posture, according to whether the surroundings of the vehicle are in a safe state.

9. The driver state recognition apparatus according to claim 7,
wherein the information arising during autonomous driving includes takeover request information for taking over manual driving from autonomous driving, and
the notification processing unit performs, if it is determined by the readiness determination unit that the driver is not in the state of being able to immediately operate the pedal of the vehicle, and the takeover request information is acquired by the information acquisition unit, notification processing for prompting the driver to take over a driving operation.

10. The driver state recognition apparatus according to claim 1,
wherein the state recognition data acquisition unit acquires, as the state recognition data, image data of a vicinity of the leg of the driver captured by a camera provided in the vehicle,
the driver state recognition apparatus further includes:
a template image storage unit configured to store a template image for inferring the state of the leg of the driver, and
the leg state inference unit infers the state of the leg of the driver through template matching performed by using the image data of the vicinity of the leg of the driver acquired by the state recognition data acquisition unit and the template image read out from the template image storage unit.

11. The driver state recognition apparatus according to claim 1,
wherein the state recognition data acquisition unit acquires, as the state recognition data, leg detection data of the driver that is detected by a sensor provided at the foot of the driver's seat of the vehicle, and
the leg state inference unit infers the state of the leg of the driver using the leg detection data of the driver acquired by the state recognition data acquisition unit.

12. A driver state recognition system comprising:
the driver state recognition apparatus according to claim 1; and
a state recognition unit configured to recognize a state of a vicinity a foot of a driver's seat of the vehicle and output the state recognition data to the state recognition data acquisition unit.

13. The driver state recognition apparatus according to claim 2, further comprising:
a notification processing unit configured to perform predetermined notification processing based on a determination result of the readiness determination unit.

14. The driver state recognition apparatus according to claim 13,
wherein, if it is determined by the readiness determination unit that the driver is not in the state of being able to immediately operate the pedal of the vehicle, the notification processing unit performs notification processing for prompting the driver to adopt an appropriate leg posture.

15. The driver state recognition apparatus according to claim 13,
wherein, if it is determined by the readiness determination unit that the driver is in the state of being able to immediately operate the pedal of the vehicle, the notification processing unit performs processing for notifying the driver that a leg posture is appropriate.

16. The driver state recognition apparatus according to claim 13,
wherein, if it is determined by the readiness determination unit that the driver is not in the state of being able to immediately operate the pedal of the vehicle, the notification processing unit performs processing for notifying an autonomous driving control apparatus that controls the autonomous driving system to continue rather than cancel autonomous driving.

17. The driver state recognition apparatus according to claim 13, further comprising:
an information acquisition unit configured to acquire information arising during autonomous driving from the autonomous driving system,
wherein the notification processing unit performs the predetermined notification processing, based on the determination result of the readiness determination unit and the information arising during autonomous driving that is acquired by the information acquisition unit.

18. The driver state recognition apparatus according to claim 17,
wherein the information arising during autonomous driving includes information for determining whether surroundings of the vehicle are in a safe state, and
the notification processing unit performs, if it is determined by the readiness determination unit that the driver is not in the state of being able to immediately operate the pedal of the vehicle, notification processing after changing a notification level for prompting the driver to correct a leg posture, according to whether the surroundings of the vehicle are in a safe state.

19. The driver state recognition apparatus according to claim 17,
wherein the information arising during autonomous driving includes takeover request information for taking over manual driving from autonomous driving, and
the notification processing unit performs, if it is determined by the readiness determination unit that the driver is not in the state of being able to immediately operate the pedal of the vehicle, and the takeover request information is acquired by the information acquisition unit, notification processing for prompting the driver to take over a driving operation.

20. A driver state recognition method for recognizing a state of a driver of a vehicle provided with an autonomous driving system, comprising:
acquiring, from a state recognition unit configured to recognize a state of a vicinity of a foot of a driver's seat of the vehicle, state recognition data of the vicinity of the foot of the driver's seat;
storing the acquired state recognition data in a state recognition data storage unit;
reading out the state recognition data from the state recognition data storage unit;
inferring a state of leg of the driver using the read state recognition data; and
determining whether the driver is in a state of being able to immediately operate a pedal of the vehicle during autonomous driving, based on inference information of the inferred state of the leg of the driver.
